(19) United States
(12) Patent Application Publication
(10) Pub. No.: US 2008/0159907 A1
(43) Pub. Date: Jul. 3, 2008

(54) Methods and apparatus for disinfecting and/or deodorizing an article

(76) Inventors: Anand G. Joshi, Bangalore (IN); Martin Zentner, Louisville, KY (US); Eric Watson, Crestwood, KY (US); Shrikant Basurtekar, Belgaum (IN); Martin Nicholas Austin, Portage, MI (US)

Correspondence Address: JOHN S. BEULICK (13307), ARMSTRONG TEASDALE LLP, ONE METROPOLITAN SQUARE, SUITE 2600, ST. LOUIS, MO, 63102-2740, US

(21) Appl. No.: 11/647,834

(22) Filed: Dec. 29, 2006

(51) Int. Cl. A61L 2/00

(52) U.S. Cl. 422/5; 422/120

(57) ABSTRACT

An apparatus includes a cabinet defining a chamber, a drawer slidably positioned within the chamber and movable between an open position and a closed position, the drawer defining a compartment configured to receive a load, in the closed position the drawer coupled to the cabinet and restricting air flow exiting the cabinet. The apparatus includes an ozone generator configured to selectively generate ozone into the compartment, an ozone conversion device configured to selectively convert ozone to oxygen, and a sensor configured to detect a level of ozone within the compartment and generate a signal representative of the level of ozone. A controller is operatively coupled to the ozone generator, the ozone conversion device and the sensor, and is configured to activate one of the ozone generator and the ozone conversion device in response to a signal received from the sensor.

### BACKGROUND OF THE INVENTION

This invention relates generally to appliances and, more particularly, to appliances for disinfecting and/or deodorizing an article.

Conventional home appliances, such as washing machines and/or drying machines disinfect and/or deodorize articles, such as clothes, by washing and drying the articles. However, such washing and drying processes take a considerable amount of time, and may only partially remove undesirable odors and/or microorganisms. For example, some odors, such as cigarette and shoe odors may not be effectively removed by the washing and drying processes. In addition, some articles, such as leather shoes and jackets, may not be washable and/or dryable to remove the undesirable odors and/or the microorganisms therefrom.

### BRIEF DESCRIPTION OF THE INVENTION

In one aspect, an apparatus is provided. The apparatus includes a cabinet defining a chamber, a drawer slidably positioned within the chamber and movable between an open position and a closed position. The drawer defines a compartment configured to receive a load. In the closed position, the drawer is configured to restrict air flow exiting the compartment. The apparatus includes an ozone generator positioned with respect to the compartment and configured to selectively generate ozone. The generated ozone is introduced into the compartment. An ozone conversion device is positioned with respect to the compartment and configured to selectively convert ozone to oxygen. A sensor is positioned with respect to the compartment. The sensor is configured to detect a level of ozone within the compartment and generate a signal representative of the level of ozone. The apparatus also includes a controller operatively coupled to the ozone generator, the ozone conversion device and the sensor. The controller is configured to activate the ozone generator and/or the ozone conversion device in response to a signal received from the sensor.

In another aspect, an appliance is provided. The appliance includes a cabinet defining a chamber. A drawer is slidably positioned within the chamber and movable between an open position and a closed position. The drawer defines a compartment configured to receive a load. In the closed position, the drawer is coupled to the cabinet and configured to restrict air from exiting the compartment. The appliance includes an ozone generator positioned within the cabinet and configured to produce ozone, and a fan positioned with respect to the compartment and configured to circulate ozone through the compartment.

In still another aspect, a method for treating a load is provided. The method includes providing an appliance including a cabinet defining a chamber. A drawer is slidably positioned within the chamber and movable between an open position and a closed position. The drawer defines a compartment configured to receive the load. The method also includes placing the load within the compartment. The drawer is moved to the closed position to seal the compartment. An ozone generator positioned with respect to the compartment is activated to selectively generate ozone. The generated ozone is introduced into the compartment to treat the load.

### BRIEF DESCRIPTION OF THE DRAWINGS

FIG. 1 is a front perspective view of an appliance including an exemplary disinfection and deodorization apparatus.

FIG. 2 is a front perspective view of the disinfection and deodorization apparatus shown in FIG. 1 with a drawer in a closed position.

FIG. 3 is a side perspective view of the disinfection and deodorization apparatus shown in FIG. 2 with a drawer in an open position.

FIG. 4 is a front perspective view of the disinfection and deodorization apparatus shown in FIG. 2.

FIG. 5 is a perspective sectional view of the disinfection and deodorization apparatus shown in FIG. 2 along a sectional line A-A.

FIG. 6 is a perspective sectional view of the disinfection and deodorization apparatus shown in FIG. 2 along a sectional line B-B.

FIG. 7 is a perspective sectional view of the disinfection and deodorization apparatus shown in FIG. 2 along a sectional line C-C.

FIG. 8 is a schematic sectional view of an exemplary flow path of the disinfection and deodorization apparatus shown in FIG. 2.

FIG. 9 is a perspective view of an alternative disinfection and deodorization apparatus.

### DETAILED DESCRIPTION OF THE INVENTION

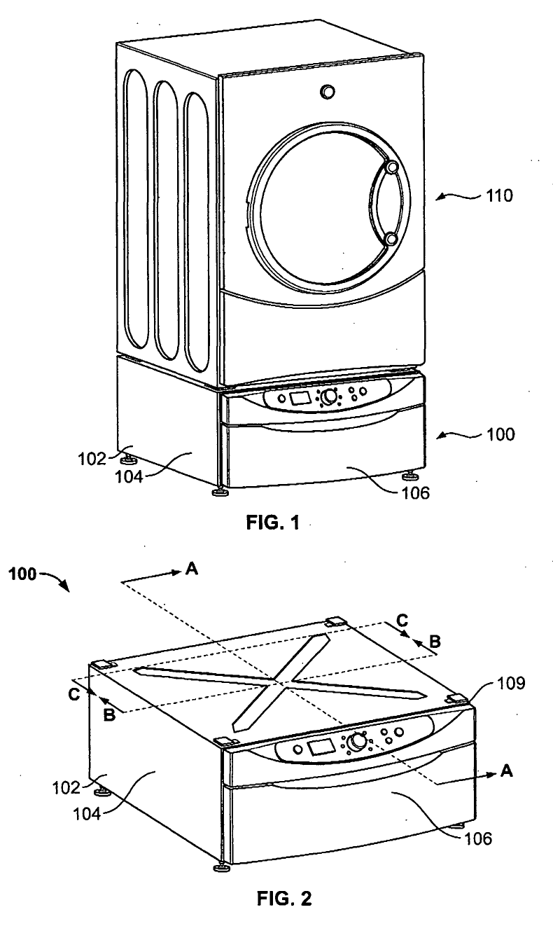

FIG. 1 is a perspective view of an exemplary disinfection and deodorization apparatus 100. As illustrated in FIG. 1, apparatus 100 is positioned below a washing machine 110 as a pedestal. Apparatus 100 includes a cabinet 102 defining a chamber 104. A drawer 106 is positioned within chamber 104. In the exemplary embodiment, drawer 106 is slidably positioned within chamber 104 and movable between a closed position, as shown in FIG. 2, and an open position, as shown in FIG. 4. It should be apparent to those skilled in the art and guided by the teachings herein provided that apparatus 100 may be used in conjunction with any suitable home or industrial appliance, such as a dishwasher, a dryer and/or a dry cleaning machine. Further, in alternative embodiments, apparatus 100 is suitable for use as a stand alone home appliance.

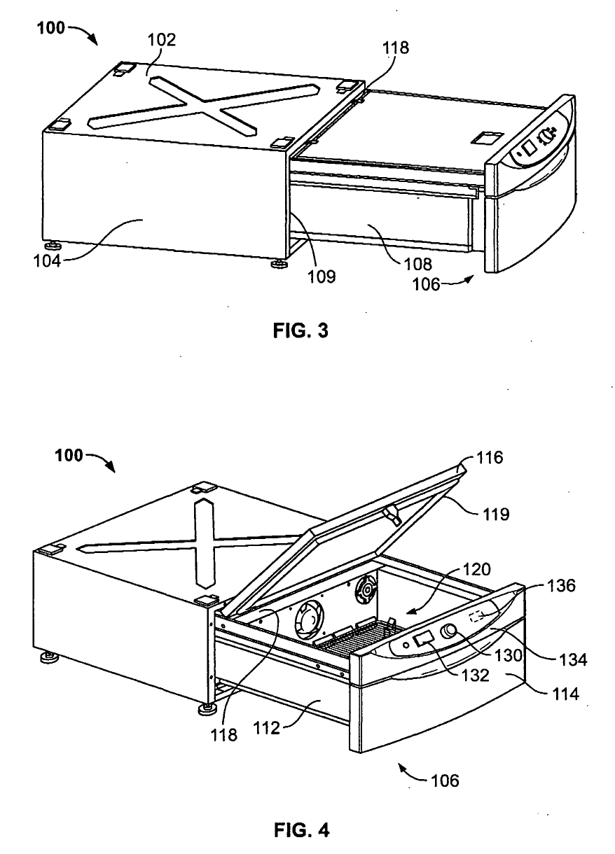

FIG. 2 is a perspective view of apparatus 100 shown in FIG. 1 with drawer 106 in a closed or retracted position. FIG. 3 is a perspective view of apparatus 100 with drawer 106 in an extended position.

In the exemplary embodiment, cabinet 102 is made of any suitable ozone compatible material including, without limitation, a metal and/or a plastic material, such as a thermoplastic material including, without limitation, a polypropylene (PP), polyvinyl chloride (PVC) and/or polycarbonate (PC) material. Drawer 106 is movable between the retracted position and the extended position. In a particular embodiment, drawer 106 includes a retracting mechanism, such as two self-retracting slide assemblies 108 mounted on opposing sides of drawer 106. As such, drawer 106 may be pulled out to the extended position, and drawer 106 retracts back to the retracted position when the pulling force is removed. Alternatively, drawer 106 is motorized to automatically move between the retracted position and the extended position. In a further embodiment, a gasket 109 is provided between cabinet 102 and drawer 106. As such, drawer 106 is sealingly coupled to cabinet 102 in the retracted position to form a substantially air-tight seal therebetween.

FIG. 4 is a perspective view of apparatus 100 shown in FIG. 2 with drawer 106 in the extended position. Drawer 106 includes a drawer frame 112 coupled to a front panel 114, and a lid 116 pivotally coupled to drawer frame 112 to cover a compartment defined within drawer 106.

In the exemplary embodiment, drawer frame 112 is made of a suitable ozone compatible material, such as a suitable metal and/or plastic material including, without limitation, PP, PVC, or PC. Lid 116 is coupled to drawer frame 112 by at least one hinge 118, and is movable between a closed position, as shown in FIG. 3, and an open position, as shown in FIG. 4. In a particular embodiment, a gasket 119 is provided on an inner surface of lid 116. As such, lid 116 is sealingly coupled to drawer frame 112 in the closed position for cooperatively forming a substantially air-tight compartment 120 defined within drawer frame 112. Lid 116 is pivotable to the open position for inserting into or removing an article, such as a sweatshirt, shoes or a jacket, from compartment 120. In this embodiment, hinge 118 is a self-standing hinge, such as a friction hinge, a spring-loaded hinge, a concealed hinge, or any suitable hinge that retains lid 116 in any desired position without user manipulation.

Front panel 114 includes a plurality of input selectors 130 and/or a display 132 mounted on an outer surface of front panel 114. Input selectors 130 and/or display 132 form a control interface 134 for user selection of operation cycles and/or operation features. Display 132 indicates the selected features and/or other items of interest to the user. A controller 136 is in operational control communication with input selectors 130 and/or display 132 for receiving from input selectors 130 and/or display 132 and/or sending to input selectors 130 and/or display 132 operational control signals. In the exemplary embodiment, controller 136 is also operatively coupled to other apparatus components to facilitate executing operation cycles and/or features, as described in greater detail below.

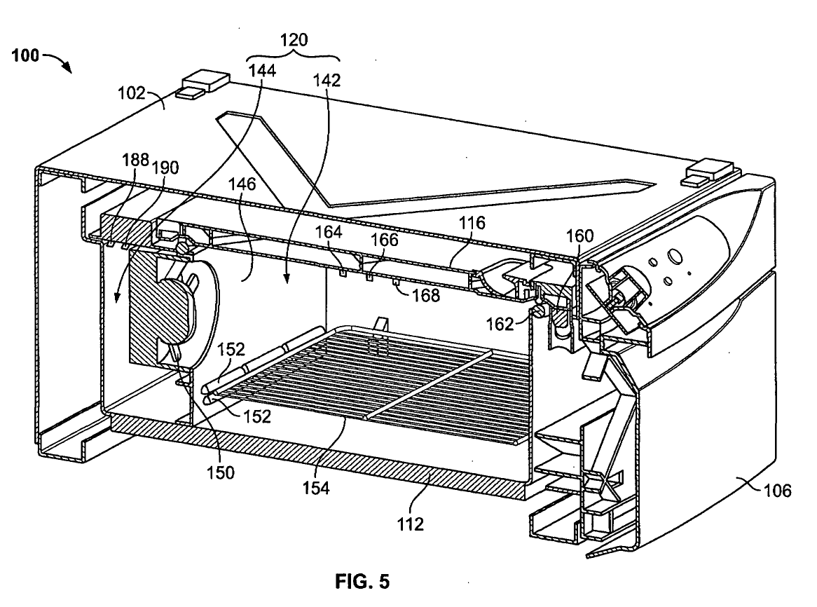

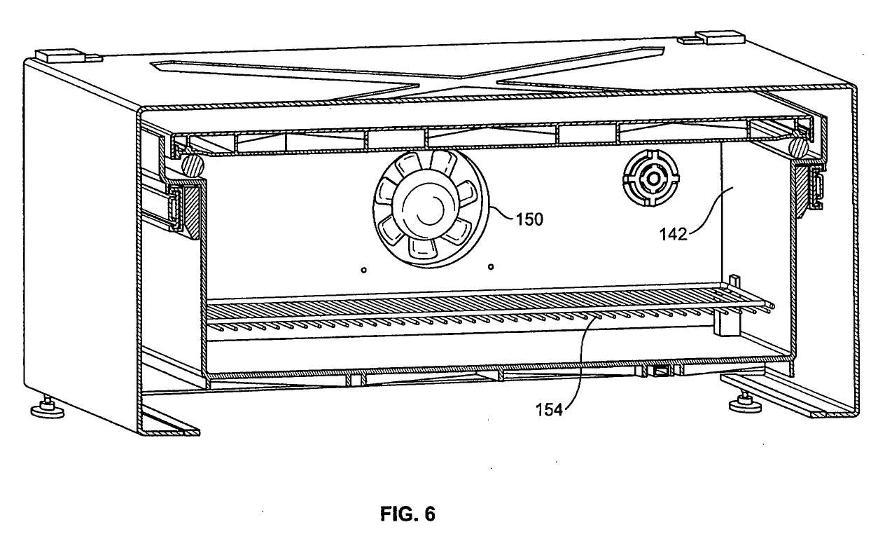

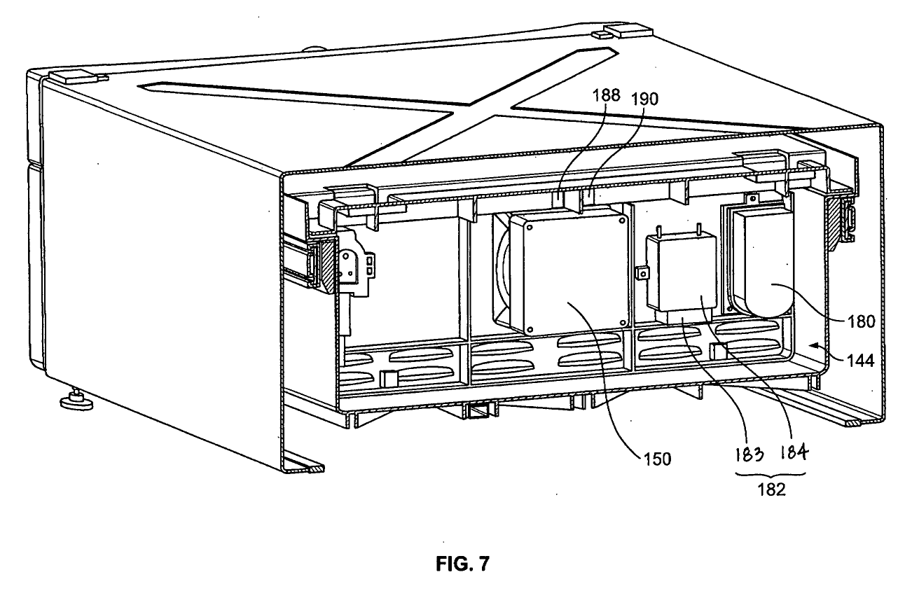

FIG. 5 is a perspective sectional view of apparatus 100 shown in FIG. 2 along a sectional line A-A. FIGS. 6 and 7 are perspective sectional views of apparatus 100 shown in FIG. 2 along sectional lines B-B and C-C, respectively.

As shown in FIG. 5, compartment 120 further includes a load compartment 142 and a machinery compartment 144 separated by a partitioning wall 146. Load compartment 142 is configured to receive articles to be treated, such as clothes, shoes, toys, carpets, cooking utensils and/or other household items. A fan 150 is positioned within compartment 120 and mounted on partitioning wall 146. Fan 150 is positioned with respect to load compartment 142 and configured to direct an air flow through load compartment 142 and machinery compartment 144. A plurality of vents 152 are defined through partitioning wall 146 to facilitate the air flow between load compartment 142 and machinery compartment 144.

A shelf 154 is positioned within load compartment 142 for supporting the articles for treatment. Shelf 154 is removable from load compartment 142 for cleaning, as desired. In a particular embodiment, a lock 160 is mounted within or positioned with respect to load compartment 142 and lid 116. In this embodiment, lock 160 is operatively coupled to controller 136, as shown in FIG. 4, such that lock 160 locks or releases lid 116 in or from, respectively, the closed position in response to an ozone level within compartment 120, as described in detail below. Lock 160 may be a motorized latch controlled by controller 136 or a magnetic lock controlled by an ozone sensor 188, described in greater detail below. It should be apparent to those skilled in the art and guided by the teachings herein provided that lock 160 may be mounted on the drawer frame 112 or lid 116 for locking lid 114. Further, lock 160 may be mounted at any suitable position within apparatus 100 to lock drawer 106 in the retracted position.

A suitable sensor 162, such as a microswitch, is positioned with respect to lid 116 for detecting the position of lid 116. Specifically, sensor 162 is operational communication with controller 136 for detecting whether lid 116 is in the closed position. In one embodiment, sensor 162 is separately provided from lock 160. In another embodiment, sensor 162 is integrated with lock 160.

In a further embodiment, load compartment 142 also includes a humidity sensor 164, a differential pressure sensor 166 and/or a perfumer 168 positioned therein. Humidity sensor 164 detects the moisture content within load compartment 142, and differential pressure sensor 166 detects the pressure within load compartment 142. Humidity sensor 164 and pressure sensor 166 are in signal communication with controller 136. Perfumer 168 is used to introduce perfume into load compartment 142, as desired, and is coupled in operational control communication with controller 136. It should be apparent to those skilled in the art and guided by the teachings herein provided that humidity sensor 164, differential pressure sensor 166, and/or perfumer 168 may be located at any suitable position within apparatus 100 to realize the desired functions.

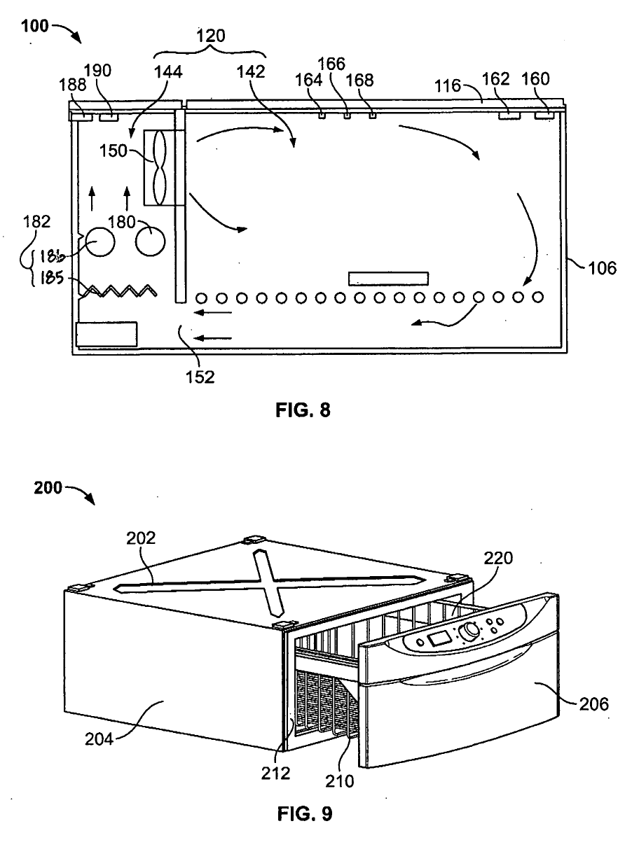

As shown in FIG. 7, machinery compartment 144 includes an ozone generator 180 and an ozone conversion device 182 positioned therein. Ozone generator 180 is used to generate ozone. In one embodiment, ozone generator 180 includes a corona discharge ozone generator or an ultraviolet lamp producible ozone, such as a 185 nm ultraviolet lamp. Ozone conversion device 182 is used to reduce the ozone within compartment 120, as desired. In the exemplary embodiment, as shown in FIG. 7, ozone conversion device 182 includes a fan and catalyst system having a fan 183 operatively positioned with respect to a catalyst material 184 to facilitate converting ozone into oxygen. In this embodiment, fan 183 is positioned with respect to catalyst material 184 to direct a flow of ozone across catalyst material 184 to facilitate converting ozone into oxygen. Catalyst material 184 may include, without limitation, a manganese compound, an active carbon and/or any suitable catalyst material known to those skilled in the art and guided by the teachings herein provided. In an alternative embodiment, as shown in FIG. 8, ozone conversion device 182 includes a heater 185 and/or an ultraviolet lamp 186, such as a 253.7 nm ultraviolet lamp, to facilitate converting ozone to oxygen. In one embodiment, heater 185 is not positioned in the direct airflow path of fan 150. In an alternative embodiment, heater 185 is positioned within the direct air flow path and/or integrated with fan 150 to facilitate converting ozone to oxygen. Ozone generator 180 and ozone conversion device 182 are coupled in operational control communication with controller 136.

An ozone sensor 188 is positioned within machinery compartment 144 for detecting a level of the ozone within load compartment 142 and/or machinery compartment 144. Ozone sensor 188 is operatively coupled to controller 136 for communicating a signal representative of the level of ozone within compartment 120. As such, controller 136 operates ozone generator 180 and/or ozone conversion device 182 in response to the signal received from ozone sensor 188. In alternative embodiments, ozone sensor 188 is located at any suitable position within apparatus 100, such as within load compartment 142 to detect the ozone level within compartment 120. In a further embodiment, a second ozone sensor 190 is provided within compartment 120 for calibrating or substituting for ozone sensor 188.

FIG. 8 is a schematic view of an exemplary air flow path within apparatus 100 shown in FIG. 2. Apparatus 100 is operatable to treat articles during a disinfection process and/or a deodorization process according to user selection.

In an exemplary disinfection process of apparatus 100, controller 136 (shown in FIG. 4) initiates the disinfection process upon position sensor 162 detecting lid 116 (shown in FIG. 4) in the closed position. Controller 136 operates lock 160 to lock lid 116 in the closed position. In the exemplary embodiment, controller 136 maintains compartment 120 at a partial vacuum level for a selected initial time period before activating ozone generator 180. During this time period, differential pressure sensor 166 monitors a change in pressure within compartment 120 to facilitate detecting a leakage situation. Upon detecting a leakage situation, controller 136 terminates the disinfection process, and visually and/or audibly prompts the user to close lid 116 properly. In one embodiment, controller 136 maintains compartment 120 in a partial vacuum condition during at least a portion of the disinfection process, and pressure sensor 166 continuously monitors for leakage. In a leakage situation, the partial vacuum condition draws outside air into compartment 120, and ozone is restricted from flowing from or exiting compartment 120. As such, the partial vacuum condition within compartment 120 facilitates preventing ozone from escaping or exiting compartment 120.

If no leakage is detected during the selected initial time period, controller 136 initiates energizing ozone generator 180 and fan 150 to execute the disinfection process. Ozone generator 180 is energized to produce ozone and fan 150 is energized to direct generated ozone into load compartment 142. Generated ozone flows across and/or through the articles within load compartment 142 to remove from the article(s) undesired odors, and/or destroys through oxidation microorganisms, such as bacteria, viruses and/or fungi. Air within load compartment 142 is circulated through load compartment 142 and into machinery compartment 144 through ozone generator 180 and/or ozone conversion device 182. As such, a treatment air flow path is created or developed through compartment 120 during the disinfection process.

In the exemplary embodiment, ozone sensor 188 detects an initial level of the ozone within compartment 120, such as an ozone level within load compartment 142 and/or an ozone level within machinery compartment 144. The user inputs a load type, including a size and/or a type of material for the article. The user also inputs a desired disinfection treatment level, such as HEAVY, MEDIUM or LOW. Controller 136 then determines a desired ozone dosage level and a treatment time based on the initial level of the ozone, the treatment level and/or the load type.

In the exemplary embodiment, controller 136 continuously maintains compartment 120 at a desired ozone level according to the determined ozone dosage level. Controller 136 energizes or de-energizes ozone generator 180 to maintain ozone within compartment 120 at the desired level. In an alternative embodiment, controller 136 continuously energizes ozone generator 180 and energizes or de-energizes ozone conversion device 182 to maintain ozone at the desired level. In a further alternative embodiment, ozone generation is time based. Controller 136 controls an operating time for ozone generator 180, a temperature within compartment 120 and/or a speed of fan 150 to maintain a desired ozone dosage level.

In a further embodiment, controller 136 also controls the ozone generation rate based at least partially on a humidity level detected by humidity sensor 164. Controller 136 sets a first time span, T1, for energizing ozone generator 180 for a first amount of time during the disinfection process. Controller 136 also sets a second time span, T2, for energizing ozone generator 180 for a second amount of time. Upon failure of ozone sensor 188, controller 136 terminates energizing ozone generator 180 when the corresponding time span T1 or T2 expires. In a particular embodiment, T1 is greater than T2.

At the end of the disinfection process, controller 136 de-energizes ozone generator 180 and energizes ozone conversion device 182 to complete the process. More specifically, fan 150 is energized to direct and circulate air from load compartment 142 to machinery compartment 144 through vents 152. Heater 185 and/or ultraviolet lamp 186 are activated to facilitate converting ozone to oxygen. Ozone sensor 188 detects whether ozone within compartment 120 is reduced to a selected safe or threshold level. In the exemplary embodiment, the threshold level is not greater than 0.1 parts per million (ppm), such as for example, 0.1 ppm, 0.08 ppm or 0.05 ppm. In a particular embodiment, second ozone sensor 190 is used for intermittent calibration of ozone sensor 188. Alternatively, second ozone sensor 190 is used as a backup sensor if ozone sensor 188 fails or is defective.

Lock 160 locks lid 116 in the closed position until the detected ozone is reduced to a safe level. As such, drawer 106 restricts generated ozone from exiting or escaping apparatus 100 during operation. In the exemplary embodiment wherein ozone conversion device 182 includes a catalyst material operatively coupled with a fan to facilitate converting ozone into oxygen, a suitable time-based algorithm is utilized to unlock lid 116 upon expiration of a suitable time period, such as about 2 minutes. In an alternative embodiment, upon detecting the ozone level below the threshold level, controller 136 terminates the disinfection process and de-energizes fan 150 and ozone conversion device 182. Lock 160 unlocks lid 116 in response to a signal from ozone sensor 188 indicating that the ozone level is below the threshold level. As such, the user may open lid 116 for retrieving the article(s) from compartment 120. In a further embodiment, controller 136 sets a maximum operation time and operates lock 160 to unlock when the maximum operation time expires. As such, controller 136 opens lid 116 if ozone sensor 188 fails or is defective.

In the exemplary deodorization process, controller 136 operates in a similar way as during the disinfection process. Controller 136 maintains compartment 120 in a partial vacuum condition to facilitate detecting a leakage situation. Controller 136 then energizes fan 150 and/or ozone generator 180 to direct generated ozone through the article(s). Ozone contacts and oxidizes undesired odor particles on and/or within the articles to remove the unwanted odor. Controller 136 also determines a desired ozone dosage and/or a treatment time based on an initial level of ozone, a desired deodorization level and in a load type. In one embodiment, controller 136 continuously maintains ozone at a desired level and/or introduces a selected amount of ozone into compartment 120 continuously or intermittently.

Upon termination of the deodorization process, controller 136 activates heater 185 and/or ultraviolet lamp 186 when an ozone level within compartment 120 is greater than a safe or threshold level. With the ozone level reduced to a safe level, controller 136 deactivates fan 150 and ozone conversion device 182 and lock 160 releases lid 116 from the closed position. In a particular embodiment, controller 136 operates perfumer 168 to introduce a desired or selected perfume onto the article(s).

FIG. 9 is a perspective view of an alternative disinfection and deodorization apparatus 200. Apparatus 200 is similar to apparatus 100, shown in FIGS. 2-8, and includes a cabinet 202 defining a chamber 204, and a drawer 206 slidably positioned within chamber 204. Drawer 206 is movable between an open position, as shown in FIG. 9, and a closed position. Drawer 206 includes a wire shelf 210 configured to receive an article or articles. In one embodiment, shelf 210 is removable for cleaning. A gasket 212 is provided between drawer 206 and cabinet 202 to sealingly coupling drawer 206 to cabinet 202 in the closed position and provide a substantially air-tight compartment 220.

In one embodiment, unlike apparatus 100, ozone sensors 188 and 190 (shown in FIG. 5) are directly mounted within chamber 204 for detecting the level of ozone within compartment 220. Humidity sensor 164, differential pressure sensor 166 and/or perfumer 168 (shown in FIG. 5) are also mounted within chamber 204 for executing the corresponding functions. Lock 160 and sensor 162 (shown in FIG. 5) are positioned with respect to drawer 206 for locking drawer 206 in the closed position and detecting the position of drawer 206, respectively. Lock 160 locks or releases drawer 206 in or from the closed position in response to a level of ozone within compartment 220. As such, drawer 206 restricts generated ozone from exiting or escaping cabinet 202 during a disinfection process and/or a deodorization process.

In the exemplary embodiment, the controller operates the ozone generator and/or the ozone conversion device to treat the article(s) in the disinfection process and/or the deodorization process. As such, articles, such as leather shoes and/or jackets, may be disinfected and/or deodorized in the apparatus to remove undesired odors. In addition, the lock operates in response to a level of ozone within the compartment to prevent residual ozone within the apparatus from exiting or escaping the apparatus.

While the invention has been described in terms of various specific embodiments, those skilled in the art will recognize that the invention can be practiced with modification within the spirit and scope of the claims.

What is claimed is:

1. An apparatus comprising:
a cabinet defining a chamber;
a drawer slidably positioned within said chamber and movable between an open position and a closed position, said drawer defining a compartment configured to receive a load, in the closed position said drawer configured to restrict air flow exiting said compartment;
an ozone generator positioned with respect to said compartment and configured to selectively generate ozone, the generated ozone introduced into said compartment;
an ozone conversion device positioned with respect to said compartment and configured to selectively convert ozone to oxygen;
a sensor positioned with respect to said compartment, said sensor configured to detect a level of ozone within said compartment and generate a signal representative of the level of ozone; and
a controller operatively coupled to said ozone generator, said ozone conversion device and said sensor, said controller configured to activate one of said ozone generator and said ozone conversion device in response to a signal received from said sensor.

2. An apparatus in accordance with claim 1 wherein said controller is configured to operate at least one of said ozone generator and said ozone conversion device to treat the load in at least one of a disinfection process and a deodorization process.

3. An apparatus in accordance with claim 1 wherein said controller is configured to operate said ozone generator based on at least one of an initial level of ozone within said compartment, a treatment level and a load type.

4. An apparatus in accordance with claim 1 further comprising a fan positioned with respect to said compartment, said fan configured to direct the generated ozone through said compartment.

5. An apparatus in accordance with claim 1 wherein said controller is configured to activate said ozone conversion device to complete at least one of a disinfection process and a deodorization process.

6. An apparatus in accordance with claim 1 wherein said ozone conversion device comprises at least one of a fan and catalyst system, a heater and an ultraviolet lamp operatively coupled to said controller.

7. An apparatus in accordance with claim 1 further comprising a lock positioned with respect to said drawer, said controller activating said lock to unlock said drawer in response to a signal from said sensor indicating that the level of ozone within said compartment is below a threshold level.

8. An appliance comprising:
a cabinet defining a chamber;
a drawer slidably positioned within said chamber and movable between an open position and a closed position, said drawer defining a compartment configured to receive a load, in the closed position said drawer configured to restrict air from exiting said compartment;
an ozone generator positioned within said cabinet and configured to produce ozone; and
a fan positioned with respect to said compartment and configured to circulate ozone through said compartment.

9. An appliance in accordance with claim 8 further comprising a fan positioned with respect to a catalyst material, said fan configured to direct a flow of ozone across said catalyst material to facilitate converting ozone to oxygen.

10. An appliance in accordance with claim 8 further comprising a heater positioned with respect to said compartment, said heater configured to heat ozone moving across said heater.

11. An appliance in accordance with claim 8 further comprising:
a sensor positioned with respect to said compartment, said sensor configured to detect a level of the ozone-within said compartment and generate a signal representative of the level of ozone; and
a controller in signal communication with said sensor and in operational control communication with said ozone generator, said controller configured to operate said ozone generator in response to the signal transmitted from said sensor.

12. An appliance in accordance with claim 11 further comprising an ozone conversion device including at least one of a fan and catalyst system, a heater and an ultraviolet lamp operatively coupled to said controller.

13. An appliance in accordance with claim 12 wherein said controller is configured to activate said at least one of said fan and catalyst system, said heater and said ultraviolet lamp to reduce the ozone within said compartment to below a threshold level.

14. An appliance in accordance with claim 11 further comprising a lock positioned with respect to said drawer and configured to unlock said drawer in response to a signal from said sensor indicating that the level of ozone within said compartment is below a threshold level.

15. An appliance in accordance with claim 11 wherein said controller is configured to maintain said compartment in a partial vacuum before activating said ozone generator.

16. A method for treating a load, said method comprising:
providing an appliance comprising a cabinet defining a chamber, a drawer slidably positioned within the chamber and movable between an open position and a closed position, the drawer defining a compartment configured to receive the load;
placing the load within the compartment;
moving the drawer to the closed position to seal the compartment;
activating an ozone generator positioned with respect to the compartment, the ozone generator configured to selectively generate ozone; and
introducing the generated ozone into the compartment to treat the load.

17. A method in accordance with claim 16 further comprising activating an ozone conversion device positioned with respect to the compartment to convert ozone to oxygen.

18. A method in accordance with claim 16 further comprising detecting a level of ozone within the compartment with a sensor positioned with respect to the compartment, the sensor configured to detect a level of ozone within the compartment and transmit to a controller in signal communication with the sensor a signal representative of the level of ozone.

19. A method in accordance with claim 18 wherein, upon detecting a level of ozone greater than a threshold level, activating an ozone conversion device positioned with respect to the compartment to convert ozone to oxygen.

20. A method in accordance with claim 19 wherein, upon detecting a level of ozone less than the threshold level, activating a lock to unlock the drawer for moving the drawer from the closed position to the open position.
